(19) United States
(12) Patent Application Publication
(10) Pub. No.: US 2012/0071757 A1
(43) Pub. Date: Mar. 22, 2012

(54) Ultrasound Registration

(75) Inventors: Septimiu SALCUDEAN, Vancouver (CA); Michael Chak Luen Yip, Sunnyvale, CA (US); Troy Kiefert Adebar, Sunnyvale, CA (US); Robert Nicholas Rohling, Vancouver (CA)

(73) Assignee: University of British Columbia, Vancouver (CA)

(21) Appl. No.: 13/235,373

(22) Filed: Sep. 17, 2011

Related U.S. Application Data
(60) Provisional application No. 61/344,706, filed on Sep. 17, 2010.

(51) Int. Cl. A61B 8/14

(52) U.S. Cl. 600/439

(57) ABSTRACT

The present invention relates to a method and apparatus for registering three-dimensional ultrasound images to an external coordinate system, such as the coordinate system attached to a camera-based imaging system or the coordinate system used to define the movements of a surgical robot. The apparatus generally comprises a tool with markers that can be accurately and repeatably located in both the ultrasound and external coordinate systems. In the case of the surgical robot frame, this tool may be a component of the surgical robot itself, such as a portion of a surgical manipulator. The method generally comprises positioning the toot upon the surface of a portion of patient tissue, imaging the tool using the ultrasound, simultaneously imaging the tool using the camera system or monitoring the current mechanical configuration of the surgical robot, localizing corresponding tool elements in both frames, and finally using the corresponding elements as registration fiducials to solve the geometric transformation that relates the two frames.

This application claims the benefit of U.S. Provisional Patent Application No. 61/344706, entitled ULTRASOUND TO CAMERA REGISTRATION, filed on Sep. 17, 2010, which is incorporated herein by reference.

### FIELD OF THE INVENTION

The present invention relates to image guidance for medical interventions in general and three-dimensional (3D) ultrasound guidance for laparoscopic and robotic-assisted surgical procedures specifically.

### BACKGROUND OF THE INVENTION

3D ultrasound has been used to create geometric models of tissue in a number of applications, including fetal and prostate imaging. In 3D ultrasound (for example, U.S. Pat. No. 5,454,371 “Method and System for Constructing and Displaying Three-Dimensional Images”) the ultrasound probe is swept over the target volume and the reflected ultrasound echoes are conveyed to a computer wherein successive two-dimensional images of the target volume are reconstructed to form a three-dimensional image of the target volume. Such a volume may be used for the segmentation of organs (for example, U.S. Pat. No. 7,162,065 “Prostate Boundary Segmentation from 2D and 3D Ultrasound Images”) that is useful for the planning of medical interventions such as prostate brachytherapy.

There have been several reports in clinical journals that have noted the benefit of intra-operative ultrasound during surgical procedures. In particular, it has been noted that intra-operative ultrasound can provide visualization and possibly outcome benefits when used to guide radical prostatectomy [O. Ukimura, T. E. Ahlering and I. S. Gill “Transrectal ultrasound-guided, energy-free, nerve-sparing laparoscopic radical prostatectomy”, Journal of Endourology, 22(9): 1993-1996, 2008].

Laparoscopic cameras, both monocular and stereoscopic, are commonly used during minimally invasive procedures to view the operative field. An example of a stereoscopic laparoscopic camera-based imaging system is the high-definition vision system used in the da Vinci Surgical System by Intuitive Surgical Inc. Standard visible-light cameras are limited by their inability to visualize subsurface anatomic features including vessels, suspicious lesions and organ or tumour boundaries. Ultrasound imaging can be applied to visualize these features. In order for such visualization to help guide surgical instruments, the view from the ultrasound imaging system should coincide with that of the camera-based imaging system. In this way, the ultrasound images appear at the correct location in the camera view during surgery.

One object of this invention is to provide a method for registering an ultrasound imaging system, specifically the coordinate system that locates the ultrasound image data in space, with an external coordinate system that locates the image viewed by a camera system.

In laparoscopic or robotic-assisted laparoscopic procedures, the surgeon is often removed from the patient and unable to directly manipulate the ultrasound transducer as needed for imaging during the procedure. A robotic system (for example, U.S. Pat. No. 7,021,173 “Remote Center of Motion Robotic System and Method”) can be used to reposition the ultrasound transducer during surgery. To minimize disruption to the work flow, it is desirable for the ultrasound transducer to be repositioned automatically so that the ultrasound images continuously contain the tips of the surgical tools. In this manner the surgeon is able to direct in which direction is the ultrasound transducer pointing or which ultrasound imaging plane is being shown on a computer display such as the surgical console by using the surgical tool as a pointer. This requires only that the position of the surgical tools be known in the coordinate system frame that locates ultrasound image data in space. In the case of laparoscopic surgery, the position of the surgical tools can be monitored in an arbitrary reference frame using an optical motion tracking system such as the Polaris system by Northern Digital Inc. or the MicronTracker system by Claron Technology Inc. Alternatively, an electromagnetic tracking system with such as the Aurora system by Northern Digital Inc. or one of the electromagnetic tracking systems by Ascension Technology Corporation can be used to monitor the location of a surgical tools. In the case of robotic-assisted surgery, the position of the surgical tools can be monitored using the kinematic reference frame defined by the robot to monitor the relative position of its linkages in space.

Another object of this invention is to provide a method for registering an ultrasound imaging system with an external coordinate system that locates laparoscopic or robotic surgical tools in space.

### BRIEF SUMMARY OF THE INVENTION

This patent application describes a technique for registering 3D ultrasound image data to an external coordinate system by identifying common points in both the 3D ultrasound and the external coordinate system.

In various embodiments of the invention, the external coordinate system may be the reference frame of a calibrated camera-based imaging system, the kinematic reference frame of a surgical robot, or the reference frame of a motion tracking system.

To indentify common points, the invention proposes to apply a registration tool to a tissue surface that can be imaged by the 3D ultrasound, so as to create ultrasound image features that can be indentified and localized in the 3D ultrasound images.

If the position and orientation of the applied registration tool in the external coordinate system and the geometric arrangement of the ultrasound features relative to the tool are known, common points can be indentified in the two frames.

In various embodiments of the invention, the position and orientation of the applied registration tool can be determined in the external coordinate system by stereo triangulation of markers on the tool, by solving the forward kinematics of a surgical robot of which one aspect is used as the registration tool, or by reading the output of a motion tracking system used to track the position of the tool.

In various embodiments of the invention the portion of the tool that is applied to generate ultrasound features may be any object that produces an identifiable intensity response in an ultrasound image, including the tips of existing laparoscopic or robotic surgical tools.

### BRIEF DESCRIPTION OF THE DRAWINGS

FIG. 1 depicts the registration of 3D ultrasound to a stereo camera using a registration tool pressed against a tissue surface.

FIG. 2 shows an example ultrasound image containing an ultrasound surface fiducial pressed against the surface of a tissue sample.

FIG. 3 depicts a possible registration tool configuration. A small plate with dimensions that allow passage through a standard laparoscopic cannula holds multiple camera and ultrasound markers.

FIG. 4 depicts a possible registration tool configuration. An expandable tool with camera and ultrasound markers can be folded to fit through a standard laparoscopic cannula.

FIG. 5 depicts a possible registration tool configuration. Camera markers are added to a standard laparoscopic tool and the tool tip is used as an ultrasound marker.

FIG. 6 depicts the registration of 3D ultrasound to a surgical robot's kinematic reference frame.

### DETAILED DESCRIPTION OF EMBODIMENTS OF THE INVENTION

Throughout the following description, an ultrasound image feature refers to an aspect of an ultrasound image that can be detected and localized in an ultrasound image. The term surface is used for sake of illustration of the invention to describe the interface between tissue and air, but it may also describe the interface between tissue and fluid, and tissue and artificial tissue, including but not limited to soft rubbers. Ultrasound image features on a tissue surface may be created in ultrasound images by impressing, for example, a spherical ball bearing (referred to in this description as a surface fiducial), a needle tip, or the tip of a pointed object. Any other object that creates a distinct hyper-echoic or hypo-echoic image feature may also be used. Metal or any other material that generates strong reflections in ultrasound is preferred. Surfaces may be roughened or otherwise processed to contain ridges that strengthen the ultrasound echo signals they generate, as done, for example, in the now discontinued Oncura EchoSeed implantable radioactive sources for Brachytherapy. Other materials and surface processing that generate strong ultrasound echoes that enhance the visibility of the fiducials may be employed according to techniques known in the art.

By image features on the tissue surface we also mean the application of a sharp object, such as a needle, through the surface. Thus image features on the tissue surface need not necessarily be exactly against the tissue surface or immediately adjacent to it, but may penetrate through the tissue surface to a known location.

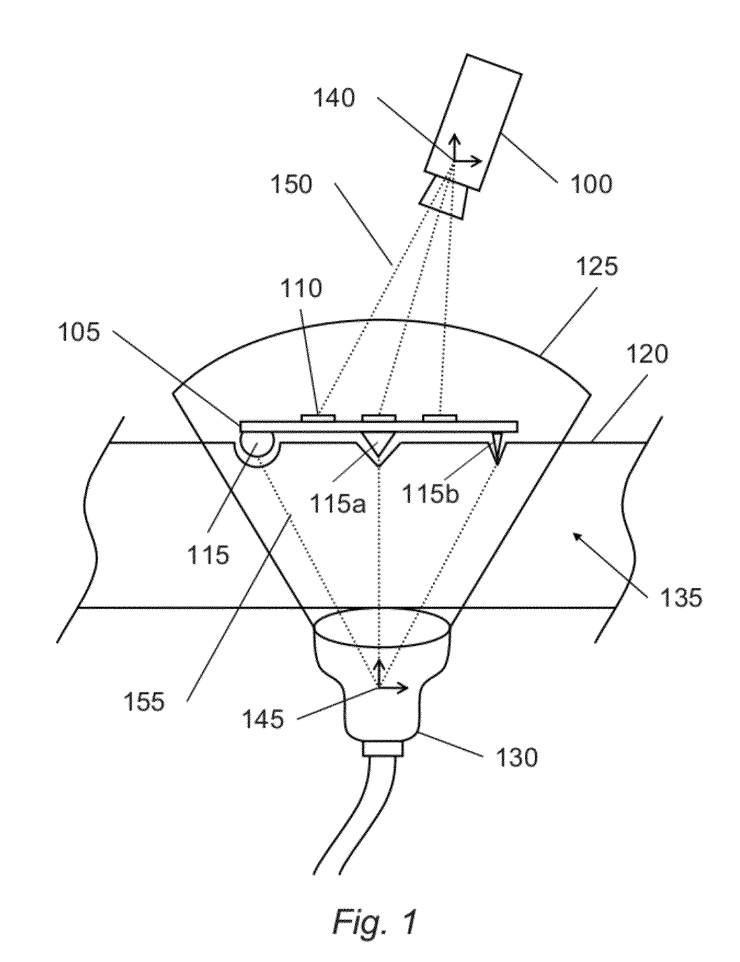

FIG. 1 illustrates one aspect of the registration invention as applied to registering 3D ultrasound to a camera-based imaging system. A camera 100 views a registration tool 105 that has embedded in it optical markers 110 and ultrasound surface fiducials 115. The registration tool 105 is pressed against an air-tissue boundary 120 so that the ultrasound surface fiducials 115 create features that are visible in an ultrasound volume 125. The ultrasound volume 125 is generated by a 3D ultrasound imaging device 130, imaging through tissue 135. Note that the features 115 can be spherical, as created by ball bearings, or pointed objects such as a conical surface 115a or a sharp needle surface 115b which may penetrate the tissue.

This arrangement is used to determine the transformation between the ultrasound coordinate system and the external camera coordinate system. A camera system 100 has a camera coordinate system 140, which can be used to define the position of the camera images in space if the optical parameters of the camera are known, according to techniques known in the field of computer vision. (See, for example, [“Calibration and orientation of cameras in computer vision”, A. Gruen and T. S. Huang, editors, Springer-Verlag, 2001, ISBN 3-540-65283-3].) The optical markers 105 which are imaged by the camera system 100 define optical marker position vectors 150 in the camera coordinate system 140. These vectors can be determined, for example, by stereo triangulation in the case of stereoscopic cameras or by model-based pose estimation in the case of monocular cameras. Furthermore, these vectors can be determined automatically with minimal or no user intervention as done, for example, in commercial systems such as the MicronTracker system by Claron Technology by known image processing techniques. The camera system may involve one, two or several cameras that produce mono or stereo vision. The camera system may involve a mono or a stereo laparoscopic camera.

Alternatively, instead of the camera system, a specialized spatial localizer system such as an optical tracker such as the Polaris system by Northern Digital Inc. or the MicronTracker system by Claron Technology Inc. Alternatively, instead of a an optical system, an electromagnetic tracking system such as the Aurora system by Northern Digital Inc. or one of the electromagnetic tracking systems by Ascension Technology Corporation can be used to monitor the location of the surgical or registration tool.

The ultrasound image features in the ultrasound volume 125 define ultrasound image feature position vectors 155 in the ultrasound coordinate system 145. These vectors can be determined by processing the ultrasound volume 125 to automatically extract the locations of the features.

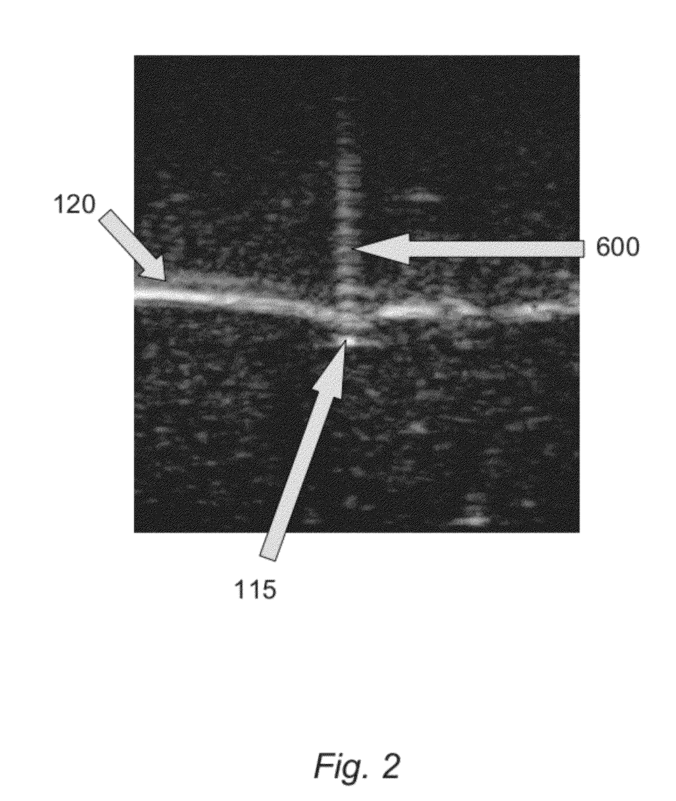

FIG. 2 shows an ultrasound image containing an example of the ultrasound image feature created by pressing an ultrasound surface fiducial 115 against a tissue surface 120. The high-intensity response near the fiducial edge, and the distinct reverberation pattern 600 creates a specific appearance in the image that makes both manual or automatic detection and localization of the fiducial possible. Techniques based on image segmentation, template matching based on a library of images, boosting, etc exists to automatically localize such these features, i.e. to determine the position vectors 155 in the ultrasound coordinate system 145. The specific detection of targets at the air-tissue boundary in ultrasound image is discussed in [T. Adebar, “A system for intra-operative trans-rectal ultrasound imaging in robotic-assisted laparoscopic radical prostatectomy”, M.A.Sc. Thesis, the University of British Columbia, August 2011], and references therein. Many other works, such as [E. J. Harris, R. Symonds-Taylor, et al., “Evaluation of a three-dimensional ultrasound localisation system incorporating probe pressure correction for use in partial breast irradiation”, British Journal of Radiology, 82, pp. 839-846, 2009] describe known methods for ultrasound image segmentation and processing in order to detect features such as 115 of FIG. 2.

Ultrasound image enhancement, for example as described in [Wen, X., S. E. Salcudean, and P. D. Lawrence, “Detection of brachytherapy seeds using 3D transrectal ultrasound”, IEEE Transactions on Biomedical Engineering, 58(10): 2467-2477, 2010] may also be employed prior to the ultrasound image segmentation and processing or prior to the manual detection of the ultrasound features by the user.

Alternatively, ultrasound features may be detected manually by a user, who may mark their location in the three dimensional ultrasound images by using a user interface to image navigation software.

The geometric relationship between the optical markers 110 and the ultrasound surface fiducials 115 is exactly known from the design geometry of the registration tool 105.

The transformation relating the ultrasound coordinate system 145 and the camera coordinate system 140 is determined as follows (adapted from [M. C. Yip, T. K. Adebar, et al., “3D ultrasound to stereoscopic camera registration through an air-tissue boundary”, Proc. MICCAI, LNCS 6362, pp. 626-634, 2010], hereby included in its entirety by reference). Three coordinate systems are defined: the camera coordinate system 140, {o₀, C₀}, the registration tool coordinate system, {o₁, C₁} attached to the registration tool, and the ultrasound coordinate system 145, {o_{c}, C_{c}}. The goal is to determine the homogeneous transformation ⁰T_{z} that relates the position and orientation (jointly called the “location”) of the ultrasound coordinate system with respect to the camera coordinate system. The optical marker position vectors 150, denoted ⁰x_{c}, and the ultrasound feature position vectors 155, denoted ²xᵤₛ, are determined as described above. The known geometric relationship between the optical markers 110 and the ultrasound surface fiducials 115 defines the offset vector ¹x_{us-c}. The offset vector ⁰x_{us-c}, defined in {o₀, C₀}, can also be determined based on ⁰x_{c}. This offset vector is used to determine the position of the ultrasound fiducials in {0₀, C₀}, ⁰xᵤₛ, as:

⁰xᵤₛ=⁰x_{c}+⁰x_{us-c} [b 1]

The vectors ⁰xᵤₛ and ²xᵤₛ then define common points known in the two coordinate systems (i.e. the centers of the ultrasound fiducials 115). Finally the transformation, ⁰T₂, can be determined by minimizing the average 3D error over ⁰T₂:

$$\frac{1}{N} {\sum }_{N \text{?}} ‖ {}_{}^{o}{x}{}_{C}^{} - {}_{}^{o}{T}{}_{2}^{1} {x}_{US} ‖ \\ \text{?} \text{indicates text missing or illegible when filed} \qquad [ 2 ]$$

This least-squares problem can be solved using standard mathematical methods. At least three common points (N=3) are needed to solve for the relationship between the ultrasound coordinate system 145 and the camera coordinate system 140. More than three common points may improve the accuracy of the registration. More than three common points can be achieved by re-positioning the registration tool 105 at several different positions and repeating the ultrasound scan. Alternatively, more than three ultrasound surface fiducials 115 can be placed on the registration tool 105.

The camera described might consist of either a stereoscopic or monocular imaging apparatus. It might be a laparoscopic camera, a flexible endoscopic camera, or any more general imaging apparatus. The three-dimensional ultrasound imaging device might consist of a 3D trans-rectal ultrasound probe, a 3D laparoscopic probe, or any other type of 3D ultrasound transducer. The transducer may obtain 3D images by scanning a linear array of transducer elements that acquire data over a volume of interest, or, alternatively, images may be generated from a 2D ultrasound array. The imaging device might also consist of a 2D imaging device with a linear array that has been motorized, tracked, or otherwise configured to collect 3D ultrasound data over a volume of interest according to current state of the art methods. An example in which the 3D images are generated from a 2D array is included in [T. A. Adebar, S. E. Salcudean, et al., “A robotic system for trans-rectal ultrasound and ultrasound elastography in radical prostatectomy”, Proc. IPCAI, LNCS 6689, pp. 79-89, 2011], which describes a modified brachytherapy stepper (EXII, CIVCO Medical Solutions) that has the ability to rotate the sagittal array of an endorectal ultrasound transducer, in order to scan the prostate and the periprostatic tissue.

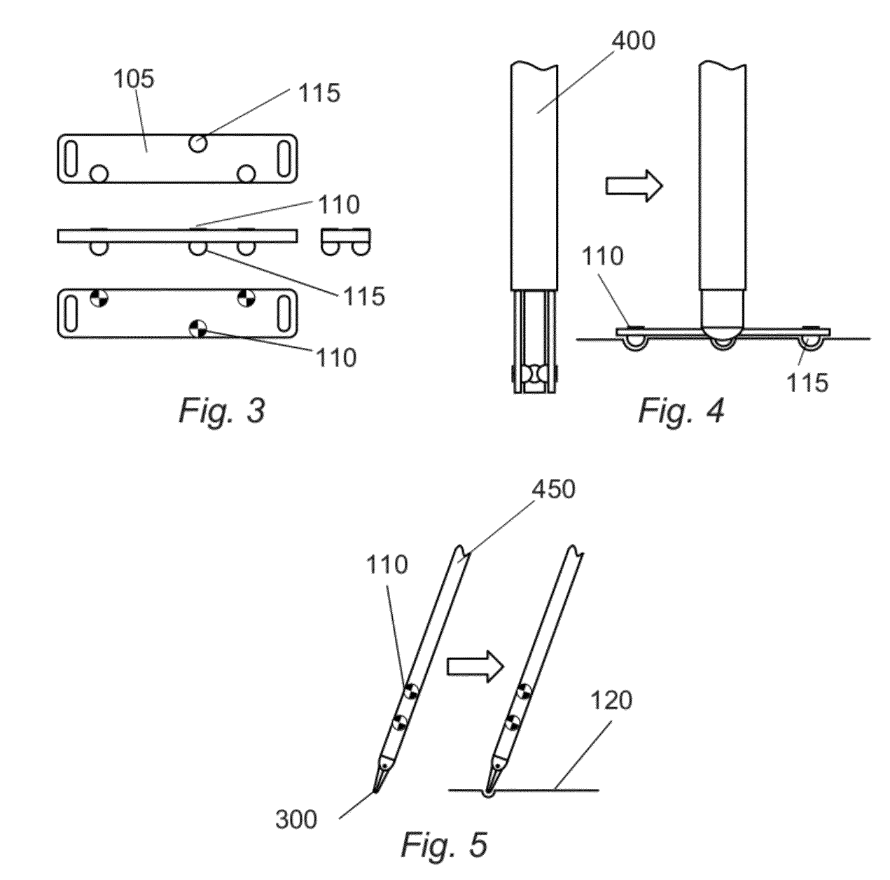

Some examples of possible implementations of the registration tool are shown in FIG. 3, FIG. 4, and FIG. 5. The tool might be implemented as a simple unarticulated probe, with both optical markers 110 and surface fiducials 115 mounted on a plate as shown in FIG. 3. The locations of the optical markers 110 are known or measured with respect to the surface fiducials 115. Another example of a possible implementation of the registration tool, not shown, is a rigid plate with both an electromagnetic position sensor and surface fiducials 115. This plate might be sized to allow the tool to pass through a surgical cannula during laparoscopic surgery. An articulated tool 400 might also be implemented, as shown in FIG. 4, so that it can pass through a surgical cannula during laparoscopic surgery, then be deployed into a larger configuration. The tool might also be implemented as a modification to an existing laparoscopic tool 450 as shown in FIG. 5 with optical markers 110 added to the body of the tool and the tip of the closed tool jaws 300 pressed against the air-tissue boundary 120 in place of the ultrasound surface fiducials. This implementation necessitates moving the tool tip to multiple positions across the surface of the air tissue boundary, capturing a new ultrasound volume at each position and combining these multiple volumes towards one registration.

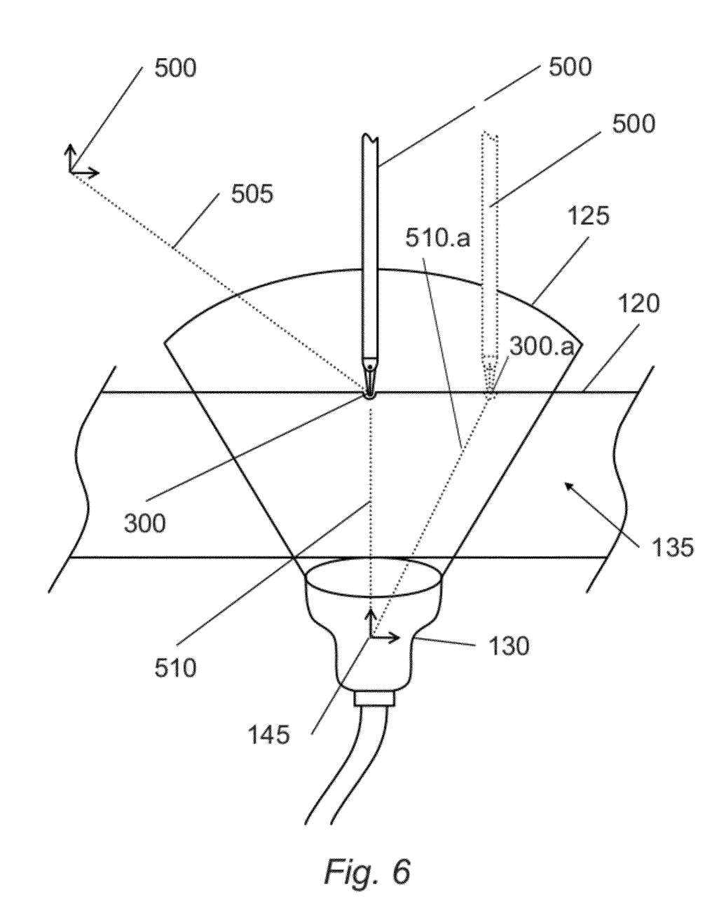

FIG. 6 illustrates one aspect of the registration invention as applied to registering 3D ultrasound to the kinematic reference frame 500 of a surgical robot, or another reference frame used to monitor the position of a surgical tool, such as the reference frame of a motion tracking system. The surgical tool tip 300 defines a tool tip position vector 505 in the kinematic reference frame 500. The location of the surgical tool tip is obtained using known techniques described in the robotics literature for forward kinematics: the sequential reconstruction of the position and orientation of serial manipulator links using the current encoder reading for each link. See [M. W. Spong, S. Hutchinson and M. Vidyasagar, Robot Modeling and Control, 2006] for a description of forward kinematics techniques.

The registration process is similar to that described above, with the exception of the camera-based imaging system. The ultrasound imaging system 130 views a tissue surface 120 by imaging through tissue 135. Like an ultrasound surface fiducial 115, the tool tip 300 can be used to create a feature in the ultrasound image. This feature can be localized in the ultrasound volume 125 to define an ultrasound position vector 510 in the ultrasound frame 145. The tool tip position vector 505 and the ultrasound position vector 510 define one common point that is known in both the kinematic reference frame 500 and the ultrasound frame 145. By moving the tool tip 300 to multiple points 300.a, etc., on the tissue surface 120, and re-imaging at each point to obtain multiple ultrasound position vectors 510.a, etc., a series of common points can be defined in the two frames. The overall transformation relating the kinematic frame 500 and the ultrasound frame 145 can be determined using known mathematical methods, as described, for example, in [M. C. Yip, T. K. Adebar, et al., “3D ultrasound to stereoscopic camera registration through an air-tissue boundary”, Proc. MICCAI, LNCS 6362, pp. 626-634, 2010] and [T. Adebar, “A system for intra-operative trans-rectal ultrasound imaging in robotic-assisted laparoscopic radical prostatectomy”, M.A.Sc. Thesis, the University of British Columbia, August 2011].

In order to improve the visibility of features at a tissue surface 120, a number of methods could be used in addition to image enhancement and segmentation as normally practiced in processing ultrasound images.

In one method, for each localization of an ultrasound feature, two ultrasound volumes can be acquired—one before the surface fiducial 115 comes in contact with the tissue-air boundary 120, and one after, with the fiducial placed at the same location. The difference between the two volumes can provide an unambiguous localization of the feature generated by the fiducial. Indeed, the application of the surface fiducial 115 will create a chance in the appearance of the ultrasound image. This change is easier to detect than when the fiducial is in a static configuration. Alternatively, the fiducial, in contact with the tissue surface, may be slightly vibrated while the ultrasound volume is acquired. This vibration, which may be generated at a known frequency, can be detected in the ultrasound image by using known tissue motion imaging techniques, such as band-pass filtering of a time series of ultrasound images as the transducer scans the 3D volume, or Doppler imaging as described in [S. H. Okazawa, R. Ebrahimi, et al., “Methods for segmenting curved needles in ultrasound images”, Med Image Anal., 10(3), pp. 330-342, 2006] and references therein, as well as in [M. P. Fronheiser, S. F. Idriss, et al., “Vibrating interventional device detection using real-time 3-D color Doppler”, IEEE Trans. Ultrason. Ferroelectr. Freq. Control, 55(6), pp. 1355-1362, 2008]. Note that the frequency vibration of the tool can be determined from processing the camera images to acquire and Fourier spectrum of the image motion.

Alternatively, the fiducial in contact with the tissue surface may be moved by the user in a manner that can be determined by digital signal processing of the images acquired by the camera 100 or that may be determined by directly monitoring the coordinates of the surgical tool tip 300 which may be computed from the robot encoders using the robot forward kinematics. Thus a trajectory of the registration tool 105 or tool tip 300 may be computed as a function of time with respect to the camera coordinate system 140 or with respect to the robot coordinate system 500. At the same time, the motion of the ultrasound tissue in the ultrasound image may also be determined as a function of time with respect to the ultrasound coordinate system 145 using well known motion tracking techniques, such as [R. Zahiri and S. E. Salcudean, “Motion estimation in ultrasound images using time-domain cross-correlation with prior estimates”, IEEE Trans. Biomed. Eng., 53(10):1990-2000, 2006]. Normalized correlation coefficients that relate tissue motion with respect to the ultrasound coordinate system 145, with registration or surgical tool motion with respect to the camera 140 or robot 500 coordinate system, may be computed and used to improve localization of the registration tool or surgical tool with respect to the ultrasound coordinate system.

An example of an application of the registration approach described above is minimally invasive radical prostatectomy, a surgical procedure that involves the removal of the prostate gland, often using a robotic system (da Vinci Surgical System by Intuitive Surgical). Benefits may accrue to this procedure from the use of a trans-rectal ultrasound system prior or during the procedure, in order to localize blood vessels, the prostate, and the bladder neck. For this purpose a typical endorectal ultrasound system consisting of an endorectal transducer mounted on the CIVCO EXII system often used in prostate brachytherapy procedures could be employed prior to and during the surgical procedure. The relationship between the endorectal transducer coordinate system and that of the da Vinci camera system could be obtained by applying the registration procedure taught in this invention description, with the 3D ultrasound volume being obtained by the endo-rectal transducer rotation of translation and the camera images of the fiducials shown in FIG. 3 being obtained by the da Vinci stereo camera system. After patient insufflation, the tissue surface anterior to the prostate can be easily imaged by both the da Vinci stereo camera system and the endorectal ultrasound transducer. A registration tool may be applied to this tissue surface. Alternatively, a surgical tool tip such as the tip of a da Vinci grasper may be applied to the tissue surface anterior to the prostate. This tool tip may be moved up and down gently by the user from the da Vinci surgical console while the surgeon rotates the endorectal ultrasound to locate the ultrasound tissue feature created by the tool tip. The ultrasound tissue feature can be pointed at and clicked with a computer input device such as a mouse, or by using one of the da Vinci masters as a pointing device. Once the registration is complete, various approaches to present the user with an “augmented reality” approach might be implemented, as described, for example, in [C. A. Linte, J. Moore, et al., “Virtual reality-enhanced ultrasound guidance: a novel technique for intracardiac interventions”, Computer Aided Surgery, 13(2), pp. 82-94, 2008].

Another example of use of this registration procedure is for partial nephrectomy procedures, in which the external 3D ultrasound placed on the patient's body may be used to image the kidney and critical structure as the surgeon is working to reach and mobilize the kidney.

Other examples of medical procedures that may benefit from this approach, using various combination of laparoscopic, external or endoscopic ultrasound with laparoscopic endoscopic or external cameras will be obvious to those skilled in the art.

1. A method for registering an ultrasound imaging system to an external coordinate system, comprising creating ultrasound image features on a tissue surface, and determining the feature positions with respect to both the ultrasound imaging system and the external coordinate system.

2. A method as in claim 1, wherein said features are created by applying a registration tool to the tissue surface.

3. A method as in claim 2, wherein said external coordinate system is the coordinate system of a camera system imaging optical markers on the registration tool.

4. A method as in claim 2, wherein said external coordinate system is the coordinate system of a motion tracking system used to monitor the location of the registration tool.

5. A method as in claim 1, wherein said features are created by applying a surgical tool to the tissue surface at one or multiple positions.

6. A method as in claim 5, wherein said surgical tool is applied by a robot and said external coordinate system is the coordinate system of the robot.

7. A method as in claim 1, wherein said ultrasound image features on a tissue surface are localized in the ultrasound images automatically based on their appearance in the image.

8. A method as in claim 1, wherein said ultrasound image features on a tissue surface are localized in the ultrasound images automatically based on a change in their appearance in the image.

9. A method as in claim 8 wherein said change in the appearance of the image occurs at a known frequency.

10. A method as in claim 8 wherein said change in the appearance in the image is correlated with the motion of a registration tool or surgical tool applied to the tissue surface.

11. A method for registering an endorectal ultrasound imaging system to a laparoscopic camera coordinate system, comprising creating ultrasound image features on a tissue surface anterior to the prostate, and determining the feature positions with respect to both the endorectal ultrasound imaging system and the laparoscopic camera coordinate system.

12. A method as in claim 11, wherein said creating of ultrasound image features comprise applying a registration tool to the tissue surface anterior to the prostate.

13. A method as in claim 11, wherein said creating of ultrasound image features comprise applying a surgical tool to the tissue surface anterior to the prostate.

14. A method as in claim 1, wherein the features are point features.

15. A method as in claim 1, wherein the features are curvilinear features.

16. A method as in claim 1, wherein the features are volumetric features.
